(19) United States
(12) Patent Application Publication
(10) Pub. No.: US 2023/0144344 A1
(43) Pub. Date: May 11, 2023

(54) COMPOSITION FOR PREVENTING OR TREATING CARDIOVASCULAR AND METABOLIC DISEASES COMPRISING ELAEOCARPUS PETIOLATUS

(71) Applicants: SEOUL NATIONAL UNIVERSITY HOSPITAL, Seoul (KR); KOREA RESEARCH INSTITUTE OF BIOSCIENCE AND BIOTECHNOLOGY, Daejeon (KR)

(72) Inventors: Hyo-soo KIM, Seoul (KR); Hyun-duk JANG, Seoul (KR); Kyung Seop AHN, Daejeon (KR); Sei-Ryang OH, Daejeon (KR); Ok-Kyoung KWON, Daejeon (KR); Hyung Won RYU, Daejeon (KR); Hang JIN, Yunnan (CN); Sangho CHOI, Daejeon (KR); Sang Woo LEE, Daejeon (KR); Jin Hyub PAIK, Daejeon (KR); Soo Yong KIM, Daejeon (KR); Changyoung LEE, Daejeon (KR); Yong In KIM, Daejeon (KR); Hyewon KIM, Daejeon (KR); Mijin PARK, Daejeon (KR)

(21) Appl. No.: 17/910,894

(22) PCT Filed: Mar. 11, 2021
(86) PCT No.: PCT/KR2021/003041

§ 371 Date: Sep. 12, 2022

(30) Foreign Application Priority Data
Mar. 11, 2020 (KR) 10-2020-0030346

(51) Int. Cl. A61K 36/185; A61K 31/7048; A61P 9/00

(52) U.S. Cl. CPC A61K 36/185; A61K 31/7048; A61P 9/00; A61K 2236/333; A61K 2236/35

(57) ABSTRACT

The present invention relates to a pharmaceutical composition for preventing or treating cardiovascular and metabolic diseases, comprising an Elaeocarpus petiolatus extract, a fraction thereof, or a compound isolated therefrom, as an active ingredient; a food composition for preventing or improving cardiovascular and metabolic diseases; and a method for preventing or treating cardiovascular and metabolic diseases using the same.

The composition comprising an Elaeocarpus petiolatus extract, a fraction thereof, or a compound isolated therefrom inhibits the binding of Resistin-CAP1 to inhibit the production of tumor necrosis factor, and thereby has an activity of inhibiting inflammation of the cardiovascular system, and thus can be effectively used as a preventive or therapeutic measure for cardiovascular and metabolic diseases.

### TECHNICAL FIELD

The present invention relates to pharmaceutical composition for preventing or treating cardiovascular and metabolic diseases, including an Elaeocarpus petiolatus extract, a fraction thereof, or a compound isolated therefrom, as an active ingredient; a food composition for preventing or improving cardiovascular and metabolic diseases; and a method for preventing or treating cardiovascular and metabolic diseases using the same.

### BACKGROUND ART

Recent abundance and diversity in diet and changes in lifestyle have tended to cause an imbalance in nutritional intake, and modern mechanized life has led to a lack of exercise. As a result, the forms of disease are also changing to those typical of advanced countries, and accordingly, the morbidity of cardiovascular and metabolic diseases is increasing. Cardiovascular and metabolic diseases refer to diseases caused by an imbalance in the metabolism of carbohydrates, lipids, etc., in vivo, and major cardiovascular and metabolic diseases include cardiovascular disease, dyslipidemia, obesity, or diabetes mellitus, etc.

Cardiovascular disease is a disease occurring in the heart and major arteries and is the leading cause of death worldwide. Major diseases belonging to cardiovascular disease include hypertension, angina, myocardial infarction, arteriosclerosis, atherosclerosis, stroke, arrhythmia, etc. Risk factors related to cardiovascular disease include age, gender, smoking, lack of exercise, obesity, etc., but the accumulation of cholesterol by lipoprotein may be considered as a representative cause when the recent westernized diet and rapid changes in lifestyle are taken into account.

Arteriosclerosis refers to a case in which the walls of blood vessels thicken due to the increased oil in the blood, the inside of the blood vessels narrows, and the lumen of the blood vessels narrows and the elasticity of the arteries decreases due the formation of blood clots. Depending on the site of occurrence, it is divided into stroke, angina, myocardial infarction, and peripheral vascular disease (Insull et al., 2009. The American Journal of Medicine 122, S3-S14). Vascular abnormalities due to atherosclerosis are an important cause of death, and the adult mortality rate caused by the disease is 50% in the United States and Japan, and 35% in Korea.

Arteriosclerosis is the accumulation of fat and fibrous tissue on the inner wall of the artery, causing narrowing or blockage of the blood vessel walls. Normal activity is not affected when arteriosclerosis is mild, but arteriosclerotic heart disease may occur when more than 50% to 70% of coronary tissue is blocked by arteriosclerosis. In severe cases, the cerebral artery or coronary artery may rupture, and cardiovascular disease such as cerebrovascular disease, heart disease, etc. develops in such cases. It is known that cerebral arteriosclerosis causes encephalomalacia, and that coronary atherosclerosis causes angina, myocardial infarction, etc. In addition, this may lead to hypertension, heart disease, cerebral hemorrhage, etc. Currently, various statin-based drugs, which are HMG-CoA reductase inhibitors, have been developed as therapeutic agents for arteriosclerosis, but there is still a need for the development of more effective therapeutic agents.

Dyslipidemia refers to a condition in which the blood contains an excess amount of lipids or fat components due to the increase in the biosynthesis of lipoproteins that transport cholesterol and triglycerides or the decrease in the degradation thereof, and as a result, it becomes a state in which total cholesterol, LDL-cholesterol, or triglycerides in the blood are increased, or a state in which HDL-cholesterol is decreased.

Dyslipidemia may be caused by genetic factors, obesity, diabetes mellitus, or drinking, etc., but in particular, a diet high in fat may increase blood lipids, and thus dyslipidemia may occur. Recently, an alternative therapy using active ingredients derived from natural products such as herbal medicines and food has been developed. However, natural pharmaceutical compositions having superior therapeutic effects and fewer side effects than conventional synthetic pharmaceutical compositions or raw materials thereof have not yet been sufficiently developed.

Obesity is widely known to cause chronic diseases such as fatty liver, hypertension, diabetes mellitus, and cardiovascular disease. According to the 2007 National Health and Nutrition Survey by the Ministry of Health, Welfare and Family Affairs, 31.7% of Korean adults are obese. In addition, 1.7 billion people, corresponding to about 25% of the world's population, are currently overweight (BMI >25), and more than 300 million people in the West, including 120 million people in major countries such as the United States, Europe, and Japan, are classified as obese (BMI >30). As an antiobestic drug sold both domestically and abroad, there is “Xenical”, containing orlistat as its main ingredient, which has been approved by the United States FDA. Xenical, which inhibits the action of lipase, is known to cause side effects in the gastrointestinal system such as fatty stool, gas production, and decreased absorption of fat-soluble vitamins, etc.

Diabetes mellitus is divided into two types: insufficient insulin secretion (type I) and impaired glucose metabolism due to insensitivity to insulin (type II). Type II is much more common, accounting for 90% of all diabetics. Type II diabetes mellitus is non-insulin-dependent diabetes mellitus/NIDDM. PPAR-γ activators, GLP-1 derivatives, DPP-IV inhibitors, PTP1 B inhibitors, etc. have so far been developed as substances for treating non-insulin-dependent diabetes mellitus, and as side effects caused by each of these, toxicity to liver, kidney, muscle, and heart, weight gain, etc. are known.

In summary, it can be said that it is important to lower the blood lipid concentration to eliminate the main causes of cardiovascular and metabolic diseases, and dietary therapy suppressing a high-fat diet, exercise therapy, and drug therapy are recommended as methods of lowering the blood lipid concentration. However, strict management and implementation of dietary therapy or exercise therapy is difficult, and there are often limitations in the effect. As lipid concentration—lowering agents developed so far, drugs that lower cholesterol content, such as bile acid-binding resins, HMG-CoA reductase inhibitors, neomycin, etc. and fibric acid derivatives, and drugs that lower the triglyceride content, such as nicotinic acid and fish oil, are used as therapeutic agents. However, these drugs have side effects such as liver toxicity, gastrointestinal disturbance, and cancer occurrence.

Resistin is known as an adipokine that induces inflammation (Bokarewa et al., 2005. Journal of immunology 174, 5789-5795; Cho, Y et al., 2011. Journal of the American College of Cardiology 57, 99-109), and is known to induce re-esterification and lipolysis of triacylglycerol stores, and to increase cholesteryl ester deposition (Rae et al., 2007. FEBS Letters 581, 4877-83).

Recently, it has been reported that resistin binds to the CAP1 protein receptor and activates NF-κB, activating the cell signaling system to induce inflammation, promoting secretion of inflammatory cytokines, such as TNF-α, thereby inducing inflammation (Lee S et al., Cell Metabolism 2014, 19(3), 484-497), and thus, compounds that inhibit the binding of Resistin-CAP1 may be used as a drug for treatment of lifestyle diseases, such as arteriosclerosis, diabetes, and especially cardiovascular and metabolic diseases.

In order to develop a therapeutic agent for cardiovascular and metabolic diseases that inhibits inflammation by inhibiting the binding of Resistin-CAP1, the inhibitory effect of Resistin-CAP1 binding was screened using an enzyme immunological method based on 100 kinds of natural materials whose anti-inflammatory activity was confirmed by the Natural Medicine Research Center of the Korea Research Institute of Bioscience and Biotechnology, and the inhibitory activity of the production of tumor necrosis factor alpha (TNF-α), an inflammatory cytokine induced by resistin, in human monocytes was screened by an enzyme immunological method.

Elaeocarpus petiolatus, which has an excellent Resistin-CAP1 inhibitory effect, belongs to the family Elaeocarpaceae, and is a plant that grows mainly in tropical, warm temperate, and temperate zones. A skin dryness and wrinkle improvement effect for anti-wrinkle agents and external preparations for skin of the same heterogeneous species Elaeocarpus sphaericus has been reported, as well as an anti-inflammatory effect on carrageenin-induced inflammation in rats. Although the antiviral effect of Elaeocarpus bifidus has been reported in the United States, studies of such plant on cardiovascular and metabolic diseases have not been conducted.

### Disclosure

### Technical Problem

Accordingly, the present researchers completed the present invention by confirming that the extracts and fractions of Elaeocarpus petiolatus and single compounds isolated therefrom inhibited Resistin-CAP1, thereby reducing the inflammatory response and thus exhibiting an effect on cardiovascular and metabolic diseases.

### Technical Solution

It is one object of the present invention to provide a pharmaceutical composition for preventing or treating cardiovascular and metabolic diseases, including an Elaeocarpus petiolatus extract, a fraction thereof, or a compound isolated therefrom, as an active ingredient.

It is another object of the present invention to provide a method for preventing or treating cardiovascular and metabolic diseases, including administering the composition to a subject.

It is still another object of the present invention to provide a food composition for preventing or improving cardiovascular and metabolic diseases, including an Elaeocarpus petiolatus extract, a fraction thereof, or a compound isolated therefrom, as an active ingredient.

### Advantageous Effects

The composition including an Elaeocarpus petiolatus extract, a fraction thereof, or a compound isolated therefrom of the present invention inhibits the binding of Resistin-CAP1 to inhibit the production of tumor necrosis factor, and thereby has an activity of inhibiting inflammation of the cardiovascular system, and thus can be effectively used as a preventive or therapeutic measure for cardiovascular and metabolic diseases.

### BRIEF DESCRIPTION OF DRAWINGS

FIG. 1 is an UPLC-QTOF-MS chromatogram of the leaf extract of Elaeocarpus petiolatus and solvent fractions.

FIG. 2a is a diagram of fractionation of butanol and water layers of the leaves of Elaeocarpus petiolatus using a column.

FIG. 2b is a chromatogram of each fraction obtained by fractionating the butanol and water layers of the leaves of Elaeocarpus petiolatus using a column.

FIG. 3 is a chromatogram of each fraction obtained by column fractionation of butanol and water layers of the leaves of Elaeocarpus petiolatus collected in large quantities.

FIG. 4a is a schematic diagram of the isolation of single components from the butanol and water layers of the leaves of Elaeocarpus petiolatus collected in large quantities.

FIG. 4b is a chromatogram of single components isolated from the butanol and water layers of the leaves of Elaeocarpus petiolatus collected in large quantities.

FIG. 5a shows the inhibitory activity of Resistin-CAP1 binding on the leaf extract of Elaeocarpus petiolatus and the solvent fractions.

FIG. 5b shows the inhibitory activity of Resistin-CAP1 binding on the column fractions obtained from the butanol and water layers of the leaves of Elaeocarpus petiolatus.

FIG. 5c shows the inhibitory activity of Resistin-CAP1 binding on treatment of single components isolated from butanol and water layer of the leaves of Elaeocarpus petiolatus collected in large quantities.

FIG. 6a is a schematic diagram of a method for measuring the inhibitory effect of Elaeocarpus petiolatus in cardiovascular and metabolic disease using Resistin-mice.

FIG. 6b is a plaque inhibitory effect of Elaeocarpus petiolatus in a cardiovascular and metabolic disease model using Resistin-mice.

FIG. 6c is an analysis of the effect of Elaeocarpus petiolatus on blood fat in a cardiovascular and metabolic disease model using Resistin-mice.

FIG. 6d shows the change in body weight of Elaeocarpus petiolatus treated group of Resistin-mice.

FIG. 7a is a chromatogram of samples of Elaeocarpus petiolatus and Elaeocarpus ganitrus.

FIG. 7b is a comparison of the TNF-α production inhibitory activity by Resistin in THP-1 cells of Elaeocarpus petiolatus and Elaeocarpus ganitrus samples.

FIG. 8 is a comparison of the TNF-α production inhibitory activity of Elaeocarpus petiolatus-derived compounds and its parent compounds.

### DETAILED DESCRIPTION OF PREFERRED EMBODIMENTS

The present invention will be described in detail below. Meanwhile, each description and embodiment disclosed herein can be applied to other descriptions and embodiments, respectively. That is, all combinations of various elements disclosed herein fall within the scope of the present invention. Further, the scope of the present invention is not limited by the specific description described below.

One aspect of the present invention to achieve the objects above provides a pharmaceutical composition for preventing or treating cardiovascular and metabolic diseases, including an Elaeocarpus petiolatus extract, a fraction thereof, or a compound isolated therefrom, as an active ingredient.

In the present invention, the Resistin-CAP1 binding inhibitory effect and the inflammatory response inhibitory effect of the Elaeocarpus petiolatus extract, a fraction thereof, or a compound isolated therefrom, which causes the onset or exacerbation of cardiovascular and metabolic diseases, were confirmed, thereby confirming that the Elaeocarpus petiolatus extract, a fraction thereof, or a compound isolated therefrom can be effectively used for the prevention, improvement, or treatment of cardiovascular and metabolic diseases.

As used herein, the term “Elaeocarpus petiolatus”, which belongs to the genus of Elaeocarpus, is an evergreen tree and shrub in tropical and subtropical regions, and is widely distributed in India, Malaysia, southern China, Japan, Australia, New Zealand, Fiji, Hawaii, etc. It is known that the Elaeocarpus petiolatus exhibits an antioxidant effect and an anti-inflammatory effect, but the effect related to cardiovascular and metabolic diseases has not yet been known, and it was first identified by the present inventors.

The leaves, stems, flowers, roots of Elaeocarpus petiolatus or a combination thereof may be used, preferably, the leaves of Elaeocarpus petiolatus may be used, but is not limited thereto.

The Elaeocarpus petiolatus may be those purchased commercially, or those harvested or cultivated in nature.

The place of origin or native habitant of the Elaeocarpus petiolatus may be China, Vietnam, Malaysia, etc., but is not limited thereto.

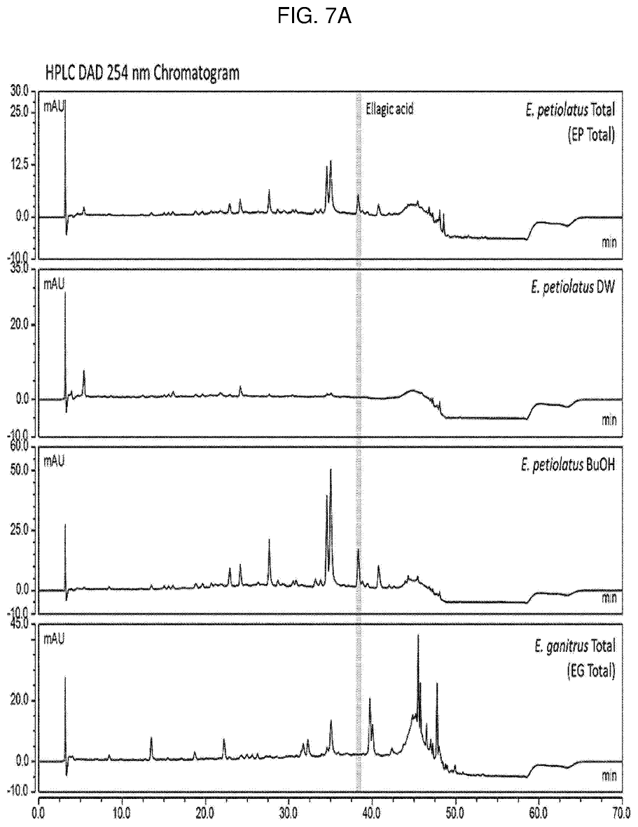

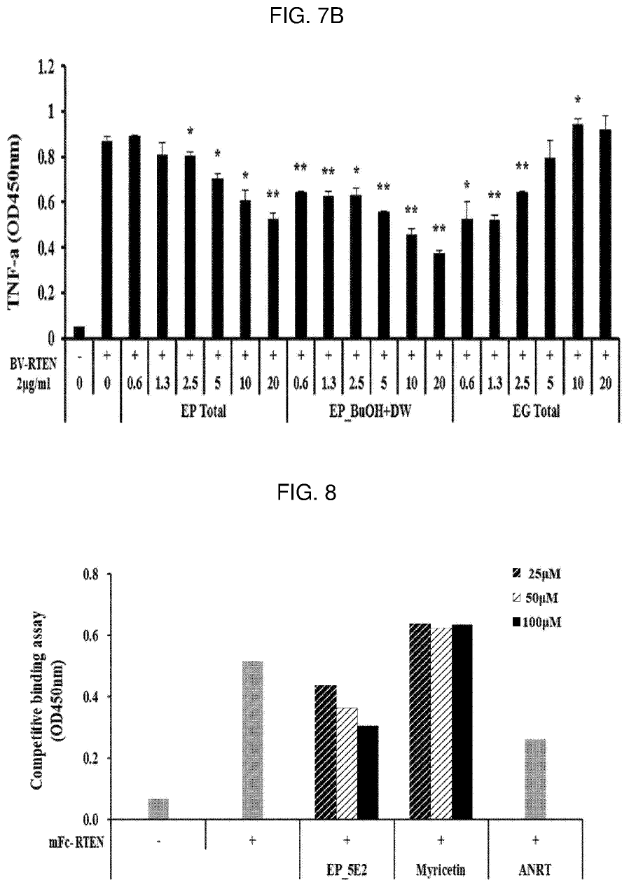

Additionally, in a specific embodiment of the present invention, as a result of comparing the TNF-α production inhibitory effect of Elaeocarpus petiolatus and Elaeocarpus ganitrus, which are the plants belonging to the genus Elaeocarpus, the TNF-α production inhibitory effect was not observed in the case of the Elaeocarpus ganitrus extract, whereas the concentration-dependent TNF-α production inhibitory effect was confirmed upon treatment with the Elaeocarpus petiolatus extract (FIG. 7b). In addition, based on the results, it was confirmed that, among the plants of the genus Elaeocarpus, especially, Elaeocarpus petiolatus can be used for the treatment of cardiovascular and metabolic diseases because it has an inhibitory activity effect of Resistin-CAP1.

As used herein, the term “extract” includes an extract solution obtained by extracting Elaeocarpus petiolatus, a diluted solution or a concentrated solution of the extract, a dried product obtained by drying the extract, a crude purification product or a purified product of the extract, or a mixture thereof, etc., and the extract itself and extracts of all formulations that can be formed using the extract.

The method of extracting Elaeocarpus petiolatus is not particularly limited, and it may be extracted according to methods conventionally used in the art. Non-limiting examples of the extraction method may include hot water extraction method, ultrasonic extraction method, filtration method, reflux extraction method, etc., and these may be performed alone or in combination of two or more methods.

In the present invention, the type of the extraction solvent used for extracting the Elaeocarpus petiolatus is not particularly limited, and any solvent known in the art may be used.

Non-limiting examples of the extraction solvent may include water, an alcohol having 1 to 4 carbon atoms, or a mixed solvent thereof, and these may be used alone or in combination of one or more thereof. Specifically, a mixed solvent of ethanol and water may be used, and the ethanol may be 10% to 100% (v/v), but is not limited thereto.

In the present invention, the Elaeocarpus petiolatus extract may be an ethanol aqueous solution extract of the leaves of Elaeocarpus petiolatus.

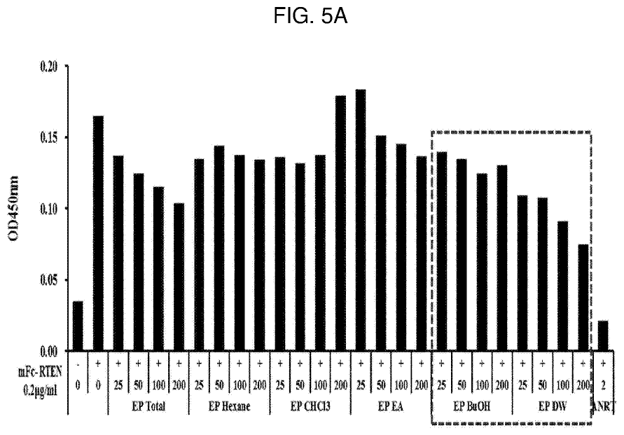

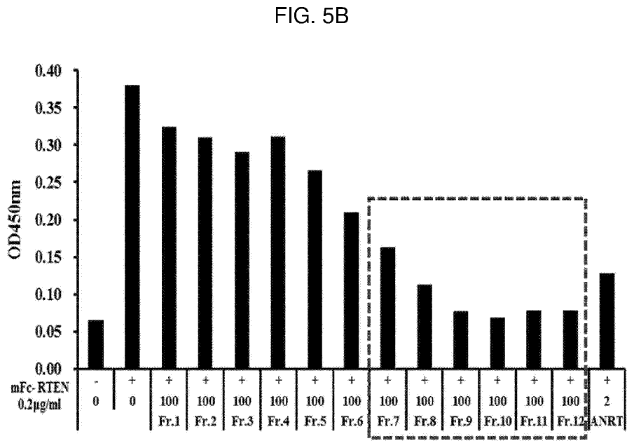

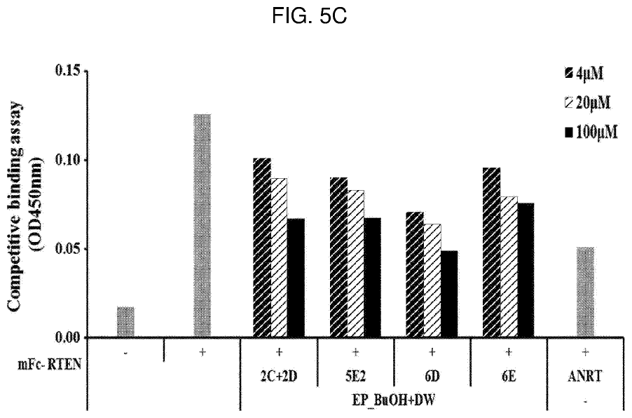

In a specific example of the present invention, the inhibitory activity of Resistin-CAP1 binding was confirmed on the extract of Elaeocarpus petiolatus (FIG. 5a), and the TNF-α production inhibitory effect by Resistin was confirmed (FIG. 7b).

Through the above results, it could be confirmed that the extract of Elaeocarpus petiolatus can be used for the treatment of cardiovascular and metabolic diseases.

As used herein, the term “fraction” refers to a resulting product obtained by performing fractionation to separate a particular component or group of particular components from a mixture containing various components.

In the present invention, the fractionation method for obtaining a fraction of Elaeocarpus petiolatus is not particularly limited and may be performed according to a method commonly used in the art. Non-limiting examples of the fractionation method may include a solvent fractionation method performed by treating various solvents, an ultrafiltration fractionation method performed by passing through an ultrafiltration membrane having a constant molecular weight cut-off value, a chromatography fractionation method performing various forms of chromatography (manufactured for separation according to size, charge, hydrophobicity, or affinity), a combination thereof, etc. Specifically, it may be a method for obtaining a fraction from the extract by treating the extract obtained by extracting of Elaeocarpus petiolatus with a predetermined solvent.

In the present invention, the type of the solvent used to obtain the fraction is not particularly limited, and any solvent known in the art can be used.

Non-limiting examples of the fraction solvents may include polar solvents such as water, an alcohol having 1 to 4 carbon atoms, etc.; non-polar solvents such as hexane, ethyl acetate, etc.; or a mixed solvent thereof. These can be used alone or in combination of one or more thereof, but is not limited thereto.

Specifically, the fraction solvent may be any one or more selected from the group consisting of hexane, chloroform (CHCl₃), ethyl acetate (EA), butanol (BuOH), and water (DW), and more specifically, butanol and water.

In addition, the extract or fraction may be prepared and used in the form of a dry powder after extraction, but is not limited thereto.

In a specific embodiment of the present invention, it was confirmed that the inhibitory activity of Resistin-CAP1 binding was confirmed on the solvent fractions of Elaeocarpus petiolatus, and in particular, it was confirmed that the binding inhibitory effect was excellent in the butanol and water layers (FIG. 5a). In addition, the inhibitory activity of Resistin-CAP1 binding was confirmed in column fractions 1 to 12 obtained by column fractionation of butanol and water layer among the solvent fractions, and in particular, it was confirmed that the column fractions 7 to 12 showed excellent effects (FIG. 5b).

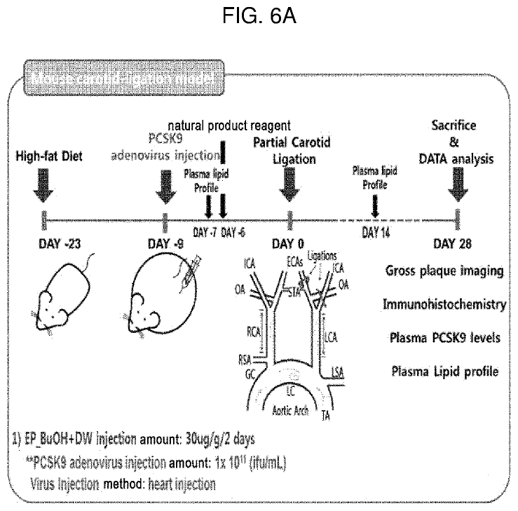

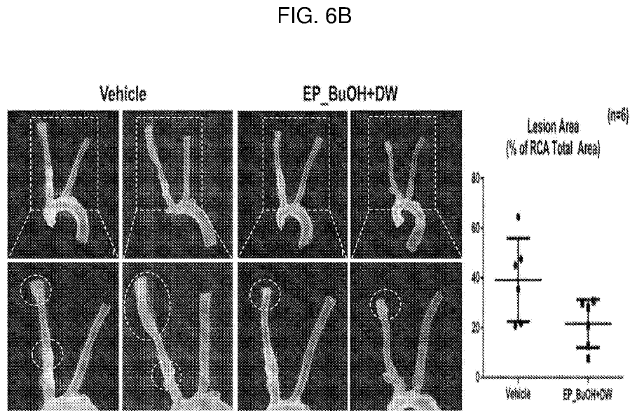

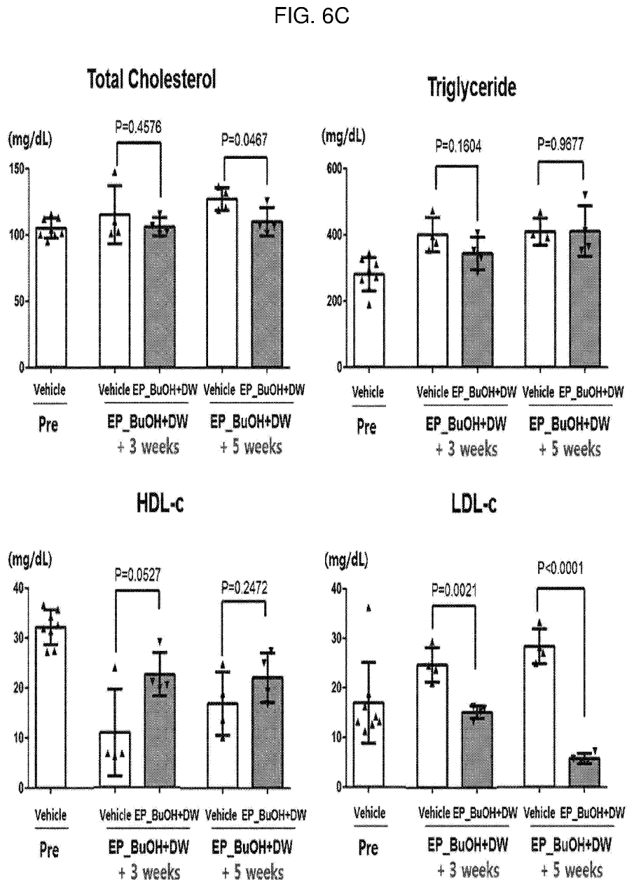

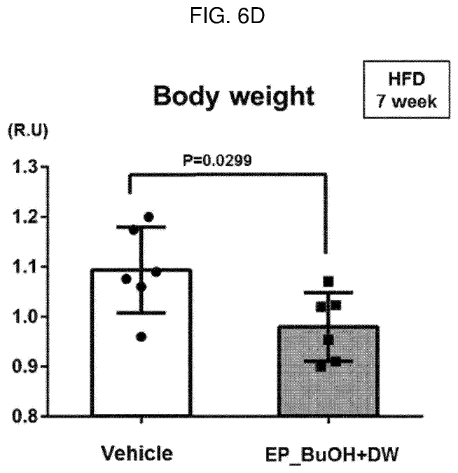

In a specific embodiment of the present invention, the plaque inhibitory effect of butanol and water layer fractions of Elaeocarpus petiolatus was confirmed in a cardiovascular and metabolic disease model (FIG. 6b), and the effect of reducing triglycerides (TG) and low-density lipoprotein cholesterol (LDL-C) in blood and the effect of increasing high density lipoprotein cholesterol (HDL-C) were confirmed (FIG. 6c).

In another specific embodiment of the present invention, the inhibitory effect of TNF-α production by Resistin of butanol and water layer fractions of Elaeocarpus petiolatus was confirmed (FIG. 7b).

Through the results as described above, it was confirmed that the fractions of Elaeocarpus petiolatus can be used for the treatment of cardiovascular and metabolic diseases.

As used herein, the term “compound isolated from Elaeocarpus petiolatus” refers to a single compound or a single substance isolated from the Elaeocarpus petiolatus, and may be obtained be by a conventional method. Specifically, a single compound can be obtained from an extract or fraction of Elaeocarpus petiolatus, and preferably, it can be obtained from a fraction using butanol and water as a fractionation solvent, but is not limited thereto.

In particular, the extract or fraction of Elaeocarpus petiolatus may be an extract or fraction obtained from the leaves of Elaeocarpus petiolatus.

The isolated compound may be methylgallate-O-hexoside, myricetin-3-O-α-L-rhamnoside, ellagic acid, isorhamnetin-3-O-β-D-hexoside, or gallic acid, but is not limited thereto.

In a specific embodiment of the present invention, the inhibitory effect of the Resistin-CAP1 binding was confirmed on the compound isolated from the butanol and water layers of the leaves of the Elaeocarpus petiolatus (FIG. 5c).

Based on the above results, it was confirmed that the compound isolated from Elaeocarpus petiolatus can be used for the treatment of cardiovascular and metabolic diseases.

As used herein, the term “cardiovascular and metabolic diseases” refers to diseases caused by an imbalance in metabolism of carbohydrates, lipids, etc. in vivo, and may include, but are not limited to, cardiovascular diseases and metabolic diseases.

The “cardiovascular disease” is a disease occurring in the heart and major arteries, and the major diseases belonging to cardiovascular disease include hypertension, angina, myocardial infarction, arteriosclerosis, atherosclerosis, stroke, arrhythmia, etc. Accumulation of cholesterol in blood vessels (increase in total cholesterol, LDL cholesterol, triglycerides, and decrease in HDL cholesterol) is one of the main causes of cardiovascular disease.

The “metabolic disease” is not particularly limited, but may include metabolic disease caused by abnormal carbohydrate metabolism or abnormal lipid metabolism. Specifically, as used herein, the “metabolic disease caused by abnormal carbohydrate metabolism” refers to a disease caused by an imbalance occurring in the metabolic process of carbohydrates in vivo, and is not particularly limited thereto, but may include diabetes mellitus, prediabetes, type II diabetes mellitus, etc. Specifically, the “metabolic disease caused by abnormal lipid metabolism” refers to a disease caused by an imbalance in the metabolic process of lipids in vivo, and is not particularly limited thereto, but may include cardiovascular disease, dyslipidemia, obesity, etc.

As used herein, the term “arteriosclerosis” refers to a disease in which blood vessels are narrowed or occluded, causing poor blood circulation to the peripheries. The arteriosclerosis may include, but is not limited to, coronary atherosclerosis and atherosclerosis. Resistin is known as a kind of adipokine that induces inflammation, and in arteriosclerosis, it is known that it induces the storage of triglycerol through re-esterification and lipolysis in human macrophages and increases the deposition of cholesteryl esters. Additionally, recently, it is known that inflammation is caused by binding to CAP1 protein, a receptor of Resistin, and activating NF-κB to activate the cell signaling system, thereby promoting the secretion of inflammatory cytokines such as TNF-α. Thus, inhibition of Resistin-CAP1 binding may play an important role in the prevention or treatment of atherosclerosis.

In a specific embodiment of the present invention, the effect on cardiovascular and metabolic diseases such as arteriosclerosis was confirmed by reducing atherosclerotic plaques, and by confirming the effect of reducing triglycerides and low-density lipoprotein cholesterol, and increasing high-density lipoprotein cholesterol, the therapeutic effect on cardiovascular and metabolic diseases was confirmed.

As used herein, the term “dyslipidemia” refers to a state in which total cholesterol, LDL-cholesterol, or triglycerides in the blood are increased, or a state in which HDL-cholesterol is decreased. Specific examples thereof include, but are not limited to, hyperlipidemia, hypercholesterolemia, or hypertriglyceridemia.

In a specific embodiment of the present invention, it was confirmed that the pharmaceutical composition had an effect on dyslipidemia by confirming the effect of reducing triglycerides, low-density lipoprotein cholesterol, and increasing high-density lipoprotein cholesterol.

In addition, the effect of reducing body weight was also confirmed, and thus the prevention effect on obesity caused by cardiovascular and metabolic diseases was also confirmed.

In the present invention, the composition including an Elaeocarpus petiolatus extract, a fraction thereof, or a compound isolated therefrom, as an active ingredient, may be one that inhibits the binding of Resistin-CAP1. The Elaeocarpus petiolatus extract, fraction, or a compound isolated therefrom exhibits an effect of inhibiting the binding of Resistin-CAP1 that causes the onset or exacerbation of cardiovascular and metabolic diseases, and shows the inhibition of NF-κB activity and an inhibitory effect on the production of TNF-α, and thus can be used for the prevention or treatment of cardiovascular and metabolic diseases.

As used herein, the term “treatment” refers to any action in which a composition including an Elaeocarpus petiolatus extract, a fraction thereof, or a compound isolated therefrom is administered to suppress or delay the onset of cardiovascular and metabolic diseases.

As used herein, the term “prevention” refers to any action in which a composition including an Elaeocarpus petiolatus extract, a fraction thereof, or a compound isolated therefrom is administered to improve or beneficially change symptoms caused by cardiovascular and metabolic diseases.

In a specific embodiment of the present invention, it was confirmed that the Elaeocarpus petiolatus extract, a fraction, or a compound isolated therefrom can inhibit the binding of Resistin-CAP1 (FIG. 5c), and the inhibitory effect of the cytokine TNF-α production was also confirmed (Table 3).

In addition, by confirming the inhibitory effect of TNF-α induced by Resistin on the sample showing the inhibitory activity of Resistin-CAP1, it was confirmed that the inhibition of TNF-α production was attributed to the inhibition of Resistin-CAP1 binding.

Further, in another specific example, the inhibitory effect of cardiovascular metabolic disease was confirmed by reducing the amount of TG and LDL in the blood and increasing the amount of HDL in the cardiovascular and metabolic disease mouse model (Resistin-mice) (FIG. 6c).

That is, by confirming the effect of inhibiting the secretion of inflammatory cytokines such as TNF-α by inhibiting the binding of Resistin-CAP, it can be implied that the composition of the present invention including an Elaeocarpus petiolatus extract, a fraction thereof, or a compound isolated therefrom can be effectively used for the prevention or treatment of cardiovascular and metabolic diseases.

The pharmaceutical composition of the present invention may contain the Elaeocarpus petiolatus extract and fraction in an amount of 0.1 μg/mL to 1000 μg/mL, specifically 0.1 μg/mL to 200 μg/mL, 0.1 μg/mL to 100 μg/mL, based on the total weight of the composition, but is not limited thereto.

The pharmaceutical composition of the present invention may contain the compound isolated from Elaeocarpus petiolatus in an amount of 0.1 g/mL to 1000 μg/mL, specifically 0.1 g/mL to 20 μg/mL, based on the total weight of the composition, but is not limited thereto.

Additionally, the pharmaceutical composition may further contain pharmaceutically acceptable carriers, excipients, and diluents, which are commonly used in the preparation of pharmaceutical compositions, and the carriers may include non-naturally occurring carriers. Specific examples of the carriers, excipients, and diluents include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, or mineral oil, but are not limited thereto.

In addition, the pharmaceutical composition of the present invention may be formulated into any one preparation selected from the group consisting of tablets, pills, powders, granules, capsules, suspensions, internal solutions, emulsions, syrups, sterilized aqueous solutions, non-aqueous solutions, suspensions, emulsions, freeze-drying agents and suppositories according to conventional methods, respectively, and may be in the form of various oral or parenteral formulations. In the case of being formulated, preparations are prepared using diluents or excipients, such as fillers, extenders, binders, wetting agents, disintegrants, and surfactants, which are commonly used. Solid preparations for oral administration include tablets, pills, powders, granules, capsules, etc., and these solid preparations are prepared by using at least one or more excipients, for example, starch, calcium carbonate, sucrose or lactose, and gelatin. In addition to simple excipients, lubricants such as magnesium stearate and talc may also be used. Liquid preparations for oral use include suspensions, internal solutions, emulsions, syrups, etc., and may contain various excipients, for example, wetting agents, sweeteners, fragrances, and preservatives in addition to water and liquid paraffin, which are commonly used simple diluents. Preparations for parenteral administration include sterile aqueous solutions, non-aqueous solutions, suspensions, emulsions, lyophilized preparations, and suppositories. As non-aqueous solvents and suspending agents, propylene glycol, polyethylene glycol, vegetable oils such as olive oil, injectable esters such as ethyl oleate, etc. may be used. As the base of suppositories, Witepsol, Macrogol, Tween 61, cacao butter, laurin butter, glycerogelatin, etc. may be used, but are not limited thereto.

Another aspect of the present invention provides a method for preventing or treating cardiovascular and metabolic diseases, including administering the composition to a subject.

In particular, the definitions of “cardiovascular and metabolic disease”, “prevention”, and “treatment” are the same as described above.

As used herein, the term “administration” refers to the introduction of the pharmaceutical composition to a subject by an appropriate method.

As used herein, the term “subject” refers to all animals including humans, rats, mice, livestock, etc., in which the cardiovascular and metabolic disease has occurred or can occur. The animal may be a mammal including not only humans but also cattle, horses, sheep, pigs, goats, camels, antelopes, dogs, cats, etc. in need of treating symptoms similar thereto, but the animal is not limited thereto.

The pharmaceutical composition of the present invention may be administered in a pharmaceutically effective amount.

The term “pharmaceutically effective amount” refers to an amount sufficient to treat diseases at a reasonable benefit/risk ratio applicable to any medical treatment. The effective dose can be determined according to factors which include the type of a subject and severity, age, sex, drug activity, sensitivity to drug, administration time, administration route and excretion rate, duration of treatment, and other drugs used simultaneously, and other factors well known in the medical field.

The pharmaceutical composition may be administered as an individual therapeutic agent or in combination with other therapeutic agents, and it may be administered sequentially or simultaneously with conventional therapeutic agents. Additionally, the pharmaceutical composition may be administered once or multiple times. Considering all of the above factors, it is important to administer an amount that can achieve the maximum effect in a minimal amount without side effects, and this can easily be determined by those skilled in the art.

Additionally, the pharmaceutical composition may be administered orally or parenterally (e.g., intravenously, subcutaneously, intraperitoneally, or topically applied) according to the desired method. The administration dose may vary depending on the patient's conditions and body weight, severity of disease, drug forms, and the route and time of administration, but it may be appropriately selected by those skilled in the art. In a specific embodiment, the pharmaceutical composition may be generally administered once or in several divided doses daily, and a preferred dose may be appropriately selected by those skilled in the art according to the conditions and weight of a subject, severity of disease, drug forms, and the route and duration of administration.

Still another aspect of the present invention provides a food composition for preventing or improving cardiovascular and metabolic diseases, including an Elaeocarpus petiolatus extract, a fraction thereof, or a compound isolated therefrom, as an active ingredient.

In particular, the definitions of “Elaeocarpus petiolatus”, “extract”, “fraction”, “isolated compound”, “cardiovascular and metabolic disease”, and “prevention” are as described above.

The food composition of the present invention can be ingested on a daily basis, and has an advantage in that there is no side effect that may occur during long-term administration of drugs since a natural substance is used as a raw material unlike general drugs, and thus can be very effectively used for the purpose of preventing or treating cardiovascular and metabolic diseases.

As used herein, the term “improvement” may refer to all actions that reduce a parameter related to the condition to be treated, for example, the degree of symptom by the consumption of the food composition.

As used herein, the term “food” includes meats, sausages, bread, chocolates, candies, snacks, confectionery, pizza, ramen, other noodles, gum, dairy products including ice cream, various soups, beverages, tea, drinks, alcoholic beverages, vitamin complexes, health functional food, health food, etc., and includes all foods in a conventional sense.

The health function(al) food is the same term as food for special health use (FoSHU), and refers to food with high medical and remedial effects processed to efficiently exhibit bioregulatory functions in addition to nutritional supply.

Herein, “function(al)” refers to obtaining useful effects for health purposes, such as regulation of nutrients or physiological actions with respect to the structure and function of the human body. The term health food refers to food having an active health maintenance or promotion effect compared to general food, and the health supplement food refers to food for the purpose of health supplementation. In some cases, the terms health functional food, health food, and health supplement food can be used interchangeably.

Specifically, the health functional food is food prepared by adding the Elaeocarpus petiolatus extract, a fraction thereof, or a compound isolated therefrom to food materials such as beverages, teas, spices, gum, and confectionery, or encapsulating, powdering, suspending the composition, which brings a specific effect on health when ingested, and has an advantage in that there is no side effect that may occur during long-term administration of drugs since food is used as a raw material unlike general drugs.

The food of the present invention may be prepared by a method commonly used in the art, and may be prepared by adding raw materials and ingredients commonly added in the art.

The form of the food composition may also not be limited as long as it is a form recognized as food. The food composition of the present invention may be prepared in various forms.

Additionally, the food composition may further contain a sitologically acceptable carrier, and the kind of carrier is not particularly limited, and any carrier commonly used in the art may be used.

The food composition may contain additional ingredients, which are commonly used in food compositions to improve odor, taste, vision, etc. The food composition may contain, for example, vitamins A, C, D, E, B1, B2, B6, and B12, niacin, biotin, folate, and pantothenic acid. Further, the food composition may contain minerals such as zinc (Zn), iron (Fe), calcium (Ca), chromium (Cr), magnesium (Mg), manganese (Mn), copper (Cu), chromium (Cr); and amino acids such as lysine, tryptophan, cysteine, valine, etc.

Additionally, the food composition may contain food additives such as preservatives (potassium sorbate, sodium benzoate, salicylic acid, sodium dehydroacetate, etc.), disinfectants (bleaching powder and high bleaching powder, sodium hypochlorite, etc.), antioxidants (butylhydroxyanisole (BHA), butylhydroxytoluene (BHT), etc.), colorants (tar pigment, etc.), color couplers (sodium nitrite, sodium nitrite, etc.), bleaching agents (sodium sulfite), seasonings (MSG sodium glutamate, etc.), sweeteners (dulcin, sodium cyclamate, saccharin, sodium, etc.), fragrances (vanillin, lactones, etc.), swelling agents (alum, D-potassium hydrogen tartrate, etc.), strengthening agents, emulsifying agents, thickeners (thickening agents), coating agents, gum bases, defoamers, solvents, and improving agents. The additives may be selected depending on the kind of food and used in appropriate amounts.

Yet another aspect of the present invention to achieve the objects provides a quasi-drug for preventing or improving cardiovascular and metabolic diseases, including an Elaeocarpus petiolatus extract, a fraction thereof, or a compound isolated therefrom, as an active ingredient

The definitions of “Elaeocarpus petiolatus”, “extract”, “fraction”, “isolated compound”, “cardiovascular and metabolic disease” and “prevention” are as described above.

As used herein, the term “quasi-drug” may be defined as a product that is used for the purposes of diagnosis, medical care, alleviation, treatment or prevention of disease in human beings or animals, excluding appliances, machinery and equipment, and a product other than an appliance, machinery or equipment that is used for the purpose of exerting pharmacological effects on the structure or functions of human beings or animals.

In the present invention, the quasi-drug composition may have an effect of preventing or improving cardiovascular disease, but is not limited thereto.

The quasi-drug composition of the present invention may further include a pharmaceutically acceptable carrier, excipient or diluent, if necessary, in addition to the above components. The pharmaceutically acceptable carrier, excipient or diluent is not limited as long as it does not impair the effects of the present invention, and may include, for example, fillers, extenders, binders, wetting agents, disintegrants, surfactants, lubricants, sweeteners, fragrances, preservatives, etc.

As used herein, the term “pharmaceutically acceptable carrier” refers to a carrier, excipient or diluent that does not cause irritation to an organism and does not abrogate the biological activity and properties of the administered compound, and may specifically be a non-naturally occurring carrier. The type of carrier usable in the present invention is not particularly limited, and any carrier commonly used in the art and pharmaceutically acceptable carriers may be used. Non-limiting examples of the carrier include saline solution, sterile water, Ringer's solution, buffered saline solution, albumin injection solution, dextrose solution, maltodextrin solution, glycerol, ethanol, etc., and these can be used alone or in a mixture of two or more thereof.

The composition containing a pharmaceutically acceptable carrier may be in the form of various oral or parenteral formulations, preferably oral formulation, but is not limited thereto. When formulated, the pharmaceutical composition is formulated using diluents or excipients, including fillers, extenders, binders, wetting agents, disintegrants, surfactants, etc., commonly used in the art. Specifically, solid formulations for oral administration include tablets, pills, powders, granules, capsules, etc. These solid formulations may be prepared by mixing at least one compound with one or more excipients, for example, starch, calcium carbonate, sucrose, lactose, gelatin, etc. In addition to simple excipients, lubricants such as magnesium stearate and talc may also be used. Liquid preparations for oral use include suspensions, internal solutions, emulsions, syrups, etc., and may contain various excipients, for example, wetting agents, sweeteners, fragrances, and preservatives in addition to water and liquid paraffin, which are commonly used simple diluents. Preparations for parenteral administration include sterile aqueous solutions, non-aqueous solutions, suspensions, emulsions, lyophilized preparations, and suppositories. As non-aqueous solvents and suspending agents, propylene glycol, polyethylene glycol, vegetable oils such as olive oil, injectable esters such as ethyl oleate, etc. may be used. As the base of suppositories, Witepsol, Macrogol, Tween 61, cacao butter, laurin butter, glycerogelatin, etc. may be used.

Examples of the quasi-drug composition of the present invention may include a disinfecting detergent, a shower foam, an ointment, a wet tissue, a coating agent, etc., but are not limited thereto. Formulation methods, dosages, methods of use, components of the quasi-drug, etc. may be appropriately selected from conventional techniques known in the art.

Even another aspect of the present invention to achieve the objects provides a use of of a composition including an Elaeocarpus petiolatus extract, a fraction thereof, or a compound isolated therefrom, as an active ingredient, for the prevention, improvement or treatment of cardiovascular and metabolic diseases.

The definitions of “Elaeocarpus petiolatus”, “extract”, “fraction”, “isolated compound”, “cardiovascular and metabolic disease”, and “prevention” are as described above.

Further another aspect of the present invention to achieve the objects provides a use of a composition including an Elaeocarpus petiolatus extract, a fraction thereof, or a compound isolated therefrom, as an active ingredient, for the preparation of medicine for the prevention or treatment of cardiovascular and metabolic diseases.

Still further another aspect of the present invention to achieve the objects provides a use of a composition including an Elaeocarpus petiolatus extract, a fraction thereof, or a compound isolated therefrom, as an active ingredient, for the preparation of food for the improvement or prevention or treatment of cardiovascular diseases.

Still further another aspect of the present invention to achieve the objects provides a use of a composition including an Elaeocarpus petiolatus extract, a fraction thereof, or a compound isolated therefrom, as an active ingredient, for the preparation of quasi-drug for the improvement or prevention or treatment of cardiovascular diseases.

### Mode for Carrying Out the Invention

Hereinafter, the composition and effect of the present invention will be described in detail by way of Examples and Experimental Examples. However, these Examples and Experimental Examples are given for illustrative purposes only, and the scope of the invention is not intended to be limited by these Examples and Experimental Examples.

### EXAMPLE 1. PREPARATION OF SOLVENT FRACTIONS OF ELAEOCARPUS PETIOLATUS AND UPLC ANALYSIS

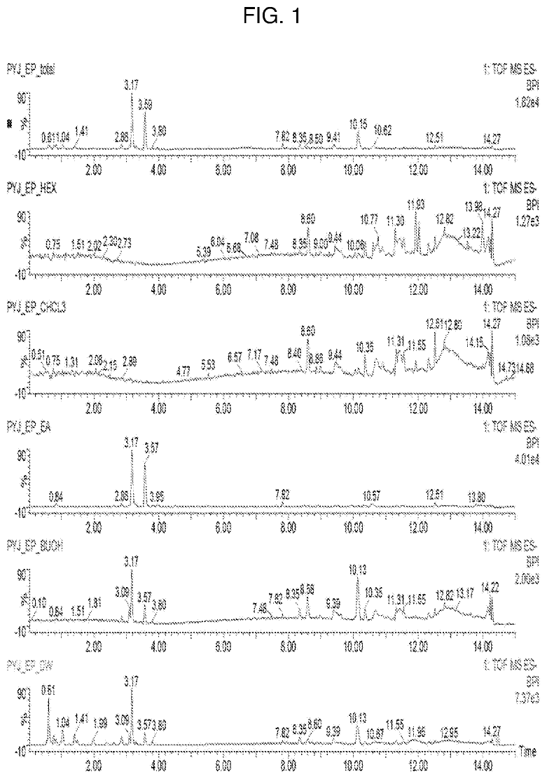

234.1 g of an extract obtained from the leaves of Elaeocarpus petiolatus in an aqueous ethanol solution was fractionated into hexane (40.3 g), chloroform (CHCl₃, 11.9 g), ethyl acetate (EA, 18.9 g), butanol (BuOH, 45.9 g), and water (DW, 95.0 g) layers to obtain a total of five fractions using a bioactive fractionation method. UPLC analysis for the qualitative analysis of the active indicator material of the leaf extract of Elaeocarpus petiolatus (extraction solvent: 95% ethanol) was performed by ACQUITY UPLC Ultra Performance equipped with a BEH C18 (2.1 mm×100 mm, 1.7 mm) column tube and a PDA detector (200-600 nm, max plot) with a flow rate of 0.4 mL/min, using distilled water containing 0.1% formic acid, and acetonitrile (see Table 1 and FIG. 1).

TABLE 1
UPLC-PDA-QTOF-MS Conditions of Leaf
Extract of Elaeocarpus petiolatus
UPLC conditions

| | A (%) | B (%) |
|---|---|---|
| Time (min) | 0.1% Formic acid/D.W. | 0.1% Formic acid/ACN |
| 0 | 90 | 10 |
| 1.0 | 90 | 10 |
| 12.0 | 2 | 98 |
| 13.4 | 2 | 98 |
| 13.5 | 90 | 10 |
| 15.0 | 90 | 10 |

QTOF Mass conditions

| Desolvation gas | N₂ | |
|---|---|---|
| Desolvation flow rate | 500 | L/h |
| Desolvation temperature. | 350° | C. |
| Source temperature | 100° | C. |
| Capillary voltage | 2300 | V |
| Con voltage | 50 | V |

| | |
|---|---|
| Scan mode | Negative |
| m/z range | 100-1500 |

### EXAMPLE 2. ISOLATION OF PURE SINGLE MATERIAL FROM LEAF EXTRACTS OF ELAEOCARPUS PETIOLATUS USING UPLC AND ANALYSIS THEREOF

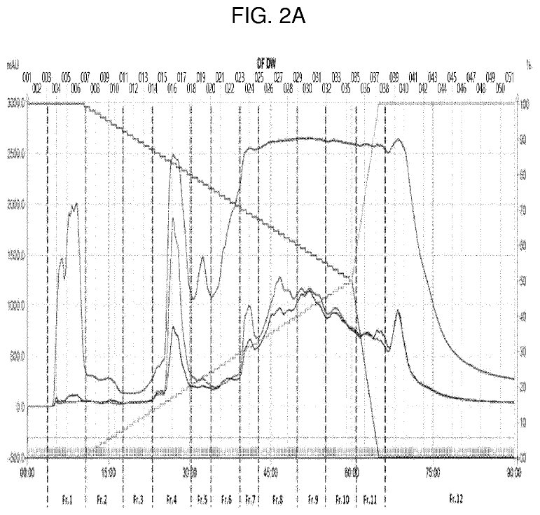

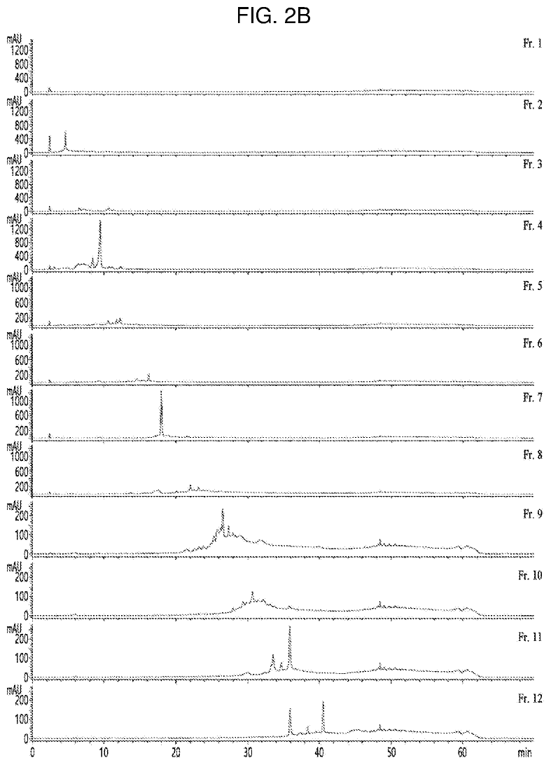

The active fractions (BuOH and water layers) showing activity among the fractions obtained in Example 1 were prepared using MPLC, and then materials exhibiting physiological activity were isolated through bioassay-guided fractionation. After loading the butanol and water (DW) layers into a column tube filled with 10 nM YMC-Pack ODS AQ-HG at 2 cm×25 cm in a SPOT PREP II 250 (MPLC, armen) device, column chromatography confirmed by a UV detector (254 nm) was performed by sequentially increasing the ratio of MeOH using MeOH—H₂O mixed solvent (0-60 min, 0-100%; 60-90 min, 100%) as a mobile phase, and a total of 12 bioactive column fractions (Fr. 1-12) were obtained through bioassay-guided fractionation (FIGS. 2A and 2B).

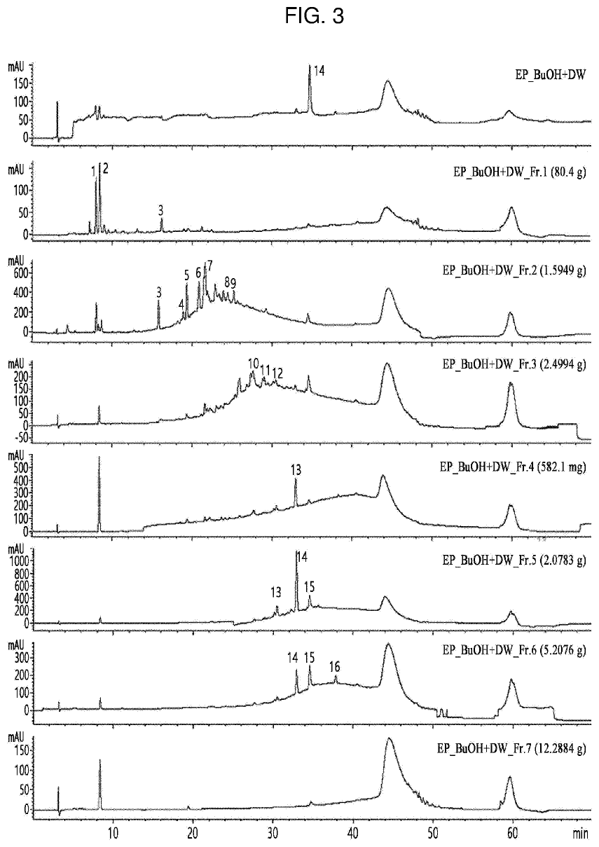

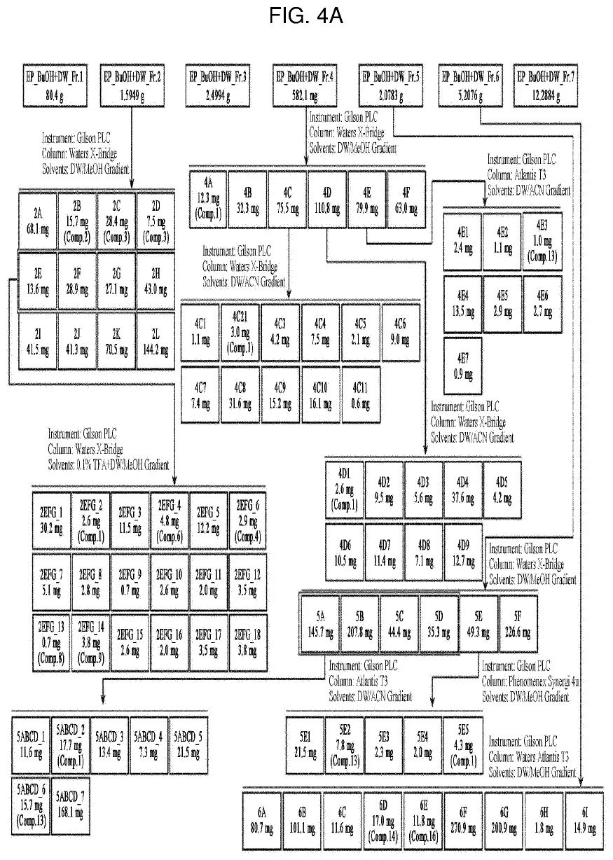

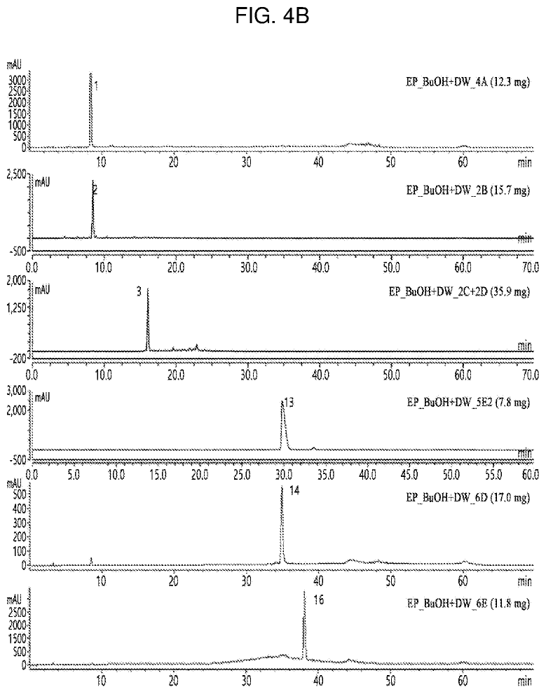

In addition, in order to isolate single materials, a large amount of Elaeocarpus petiolatus leaves was collected to obtain a butanol layer (BuOH) and a water layer (DW), and from this, it was divided into seven fractions through a column and analyzed through HPLC (FIG. 3). Based on this, single materials were isolated by the method of FIG. 4A. The isolated single materials were six types of compounds, and the names of the compounds were determined through spectroscopic data (Table 2), and the chromatograms of the isolated compounds are shown in FIG. 4B.

TABLE 2
Spectroscopic Data of 6 Compounds Isolated from BuOH and DW Layers
of Elaeocarpus petiolatus Collected in Large Quantities.

| | | UV | | |
|---|---|---|---|---|
| | | Rt | UV | Name of |
| Peak | Fraction | (min) | (nm) | Compounds |
| Peak 1 | EP_BuOH + DW_4A | 3.48 | 216, 271 | Gallic acid |
| Peak 2 | EP_BuOH + DW_2B | 3.48 | 221, 271 | Gallic acid |
| Peak 3 | EP_BuOH + DW_2C | 3.95 | 221, 274 | Methylgallate-O- |
| | EP_BuOH + DW_2D | | | hexoside |
| Peak 13 | EP_BuOH + DW_5E2 | 5.21 | 231, 259, | Myricetin-3-O-α- |
| | | | 350 | L-rhamnoside |
| Peak 14 | EP_BuOH + DW_6D | 5.15 | 255, 367 | Ellagic acid |
| Peak 16 | EP_BuOH + DW_6E | 5.55 | 226, 263, | Isorhamnetin-3- |
| | | | 331 | O-β-D-hexoside |

### EXAMPLE 3. RESISTIN-CAP1 BINDING INHIBITORY ACTIVITY (RESISTIN-CAP1 COMPETITIVE BINDING ASSAY)

In order to determine whether the leaf extracts and fractions of Elaeocarpus petiolatus directly inhibited the Resistin-CAP1 binding, the extracts and fraction samples were confirmed using an enzyme immunological method.

Recombinant CAP-1 protein was attached to a plate for immunoassay overnight, washed, and then the attachment of other proteins was blocked with 1% BSA/PBS solution, and the recombinant Resistin to which mouse Fc was attached and the sample were pre-reacted for 1 hour. Then, the solution was dispensed on the plate and reacted at room temperature for 2 hours. After washing the plate, HRP to be attached to the mFc of Resistin bound to CAP-1 was dispensed and reacted for 1 hour. After washing, the substrate was dispensed and reacted for 30 minutes by HRP, and absorbance was measured at a wavelength of 450 nm with a microplate meter.

The absorbance of the group in which mFc-Resistin was bound to CAP-1 was taken as 100%, and the inhibition rate of Resistin-CAP1 binding by the compound was calculated.

As a result, in the case of the extract of Elaeocarpus petiolatus, it was confirmed that the Resistin-CAP1 binding inhibitory effect was excellent (FIG. 5a).

In addition, it was confirmed that among the solvent fractions of Elaeocarpus petiolatus, the butanol and water layers had the most superior Resistin-CAP1 binding inhibitory effect (FIG. 5a).

When the bioactive column fractions of the butanol and water layers were used as a sample, it was confirmed that the column fractions corresponding to Nos. 7-12 out of 13 column fractions exhibited a strong Resistin-CAP1 binding inhibitory effect (FIG. 5b).

In addition, it was confirmed that the single compounds isolated from the butanol and water layer fractions obtained by collecting a large amount of Elaeocarpus petiolatus leaves also showed the effect of Resistin-CAP1 binding inhibitory activity, and it was confirmed that a concentration-dependent inhibitory effect of Resistin-CAP1 was observed in all of single compounds of methylgallate-O-hexoside, myricetin-3-O-α-L-rhamnoside, ellagic acid, or isorhamnetin-3-O-β-D-hexoside (FIG. 5c).

As a result, the inhibitory effect of Resistin-CAP1 binding of the Elaeocarpus petiolatus extracts, fractions, and single compounds isolated therefrom was confirmed.

### EXAMPLE 4. INHIBITORY ACTIVITY OF CYTOKINE PRODUCTION

TNF-alpha assay was performed to examine the anti-inflammatory effect induced by the Resistin-CAP1 binding on the Elaeocarpus petiolatus extracts, fractions, and single compounds isolated therefrom.

THP-1 cells (human monocytes) were cultured in RPMI (Welgene, Korea) medium supplemented with 10% Fetal Bovine Serum (FBS), and the inhibition rate of the sample on Resistin-induced TNF-α production was measured. THP-1 cells were suspended at a concentration of 5×10⁵ cells/mL and inoculated at 100 μL in a 96-well plate, and then each sample was treated at a concentration of 5 μM. After culturing for 1 hour, 2 μg/mL of human recombinant Resistin (Biovision) was treated and further cultured for 6 hours. Thereafter, the supernatant was recovered and stored at −70° C.

For TNF-alpha assay, Human TNF-alpha ELISA kit (BD bioscience) was used and analyzed according to the manufacturer's protocol.

In FIG. 5b of Example 3, the inhibitory activity of TNF-α induced by Resistin for Nos. 7 to 12, which had superior effect among 12 column fractions of butanol and water layers of Elaeocarpus petiolatus, in which the binding inhibitory activity of Resistin-CAP1 was confirmed, was measured (Table 3a).

As a result, as shown in Table 3a, it was confirmed that all of the fractions exhibited an inhibitory effect of 50% or more when treated at a concentration of 10 μg/mL.

In addition, the inhibitory effect on TNF-α induced by Resistin on single compounds isolated from the butanol and water layers secured by mass collection of Elaeocarpus petiolatus was confirmed. As a result, it was confirmed that all three compounds (myricetin-3-O-α-L-rhamnoside, ellagic acid, and isorhamnetin-3-O-β-D-hexoside) exhibited an inhibitory effect on TNF-α production (Table 3b).

Based to the above results, the inhibitory activity of TNF-α was confirmed through the inhibition of the Resistin-CAP1 binding of the Elaeocarpus petiolatus extracts, fractions, and single compounds isolated therefrom.

TABLE 3a
TNF-α Inhibitory Effect of Butanol and Water
Layer Column Fractions of Elaeocarpus petiolatus

| | | | TNF-α Inhibition Rate (%, |
|---|---|---|---|
| Samples | μg/mL | RETN | Average ± Standard Deviation) |
| Butanol and | 10 | + | 61.52 ± 4.40 |
| Water Layer | | | |
| Fr. 7 | 10 | + | 76.48 ± 1.98 |
| Fr. 8 | 10 | + | 83.85 ± 4.01 |
| Fr. 9 | 10 | + | 84.23 ± 2.60 |
| Fr. 10 | 10 | + | 73.76 ± 6.84 |
| Fr. 11 | 10 | + | 79.20 ± 0.51 |
| Fr. 12 | 10 | + | 70.19 ± 1.51 |

TABLE 3b
TNF-α Inhibitory Effect of Compounds
Isolated from Elaeocarpus petiolatus

| | | | TNF-α Inhibition Rate (%, |
|---|---|---|---|
| Samples | μg/mL | RETN | Average ± Standard Deviation) |
| Myricetin-3-O- | 5 | + | 28.57 ± 6.79 |
| α-L- | 10 | + | 24.13 ± 3.31 |
| rhamnoside | 20 | + | 33.64 ± 9.21 |
| (5E2) | | | |
| Ellagic acid | 5 | + | 16.40 ± 5.12 |
| (6D) | 10 | + | 27.44 ± 4.50 |
| | 20 | + | −18.61 ± 3.16 |
| Isorhamnetin- | 5 | + | 43.06 ± 10.44 |
| 3-O-β-D- | 10 | + | 51.29 ± 5.76 |
| hexoside (6E) | 20 | + | 59.53 ± 9.02 |

### EXAMPLE 5. INVESTIGATION OF CELL VIABILITY

In order to confirm the effect of the fractions of Elaeocarpus petiolatus and the compounds sample isolated therefrom on the cell viability, THP-1 cells were suspended at a concentration of 1×10⁵ cells/mL and inoculated into a 96-well plate at 100 μL. After 1 hour, each sample was treated at a concentration of 5 μM. After culturing for 24 hours, 5 μL of CytoX (LPS solution, Korea) solution was added into each well and incubated for another 4 hours. Then, absorbance was measured at 450 nm. The cell viability was calculated according to the following equation with the value of the negative control treated with DMSO as 100%.

As a result, among the fractions for butanol and water layer of Elaeocarpus petiolatus and 12 column fractions for butanol and water layers, it was confirmed that column fractions 7 to 12, which had an excellent inhibitory effect of Resistin-CAP1 binding, did not affect the cell viability at the concentration of 10 μg/mL, respectively (Table 5a).

TABLE 5a
Effect of Butanol and Water Column Fractions of
Elaeocarpus petiolatus on Cell Viability

| | | Viability (%, Average ± |
|---|---|---|
| Samples | μg/mL | Standard Deviation) |
| Control | 0 | 100.00 ± 3.86 |
| BuOH + DW | 10 | 101.62 ± 3.15 |
| Fr. 7 | 10 | 104.17 ± 6.51 |
| Fr. 8 | 10 | 106.07 ± 7.45 |
| Fr. 9 | 10 | 102.85 ± 6.06 |
| Fr. 10 | 10 | 99.36 ± 3.27 |
| Fr. 11 | 10 | 101.70 ± 3.67 |
| Fr. 12 | 10 | 99.33 ± 3.08 |

In addition, as a result of examining the effect of the single compounds isolated from the butanol and water layer of the mass-collected Elaeocarpus petiolatus on the viability of THP-1 cells, as shown in Table 5b, it was confirmed that they did not significantly affect the cell viability at a concentration of 2.5-20 μg/mL (Table 5b).

TABLE 5b
Effect of Compounds Isolated from Elaeocarpus petiolatus
on Cell Viability

| | | Viability (%, Average ± |
|---|---|---|
| Samples | μg/mL | Standard Deviation) |
| Control | 0 | 100.00 ± 0.93 |
| Myricetin-3- | 2.5 | 99.54 ± 3.22 |
| O-α-L- | 5 | 106.14 ± 0.93 |
| rhamnoside | 10 | 98.85 ± 1.07 |
| (5E2) | 20 | 100.12 ± 0.64 |
| Ellagic acid | 2.5 | 96.89 ± 0.05 |
| (6D) | 5 | 98.73 ± 0.91 |
| | 10 | 94.38 ± 0.13 |
| | 20 | 89.21 ± 1.01 |
| Isorhamnetin- | 2.5 | 101.71 ± 4.56 |
| 3-O-β-D- | 5 | 101.88 ± 0.10 |
| hexoside (6E) | 10 | 95.02 ± 0.20 |
| | 20 | 94.72 ± 0.28 |

### EXAMPLE 6. INHIBITORY EFFECT OF ELAEOCARPUS PETIOLATUS ON CARDIOVASCULAR AND METABOLIC DISEASES IN RESISTIN-MOUSE MODEL

### EXAMPLE 6-1. CONSTRUCTION OF CARDIOVASCULAR AND METABOLIC DISEASE MODEL MOUSE (RESISTIN-MOUSE)

In order to construct a cardiovascular and metabolic disease model mouse, an atherosclerosis model was prepared by inducing inflammation, thrombosis, oxidative stress, and shear stress by causing mechanical stress through ligation of the carotid artery of 6-8-week-old Resistin-mice and changing the function and structure of external carotid artery (EC) (Cho et al., 2011).

Specifically, carotid artery ligation was performed on rats fed with a high-fat diet for one week, and ligation was performed on the left common carotid artery (LCA) after abdominal anesthesia. The three branches in the LCA, the external carotid artery (ECA), the internal carotid artery (ICA), and the occipital artery (OA) were partially ligated, and at this time, the ICA and OA were ligated, and ECA was ligated separately. In mice subjected to ligation, the amount of blood flow to the heart was reduced and the direction thereof changed, thereby allowing the construction of an atherosclerosis model (FIG. 6a).

### EXAMPLE 6-2. CONFIRMATION OF CARDIOVASCULAR AND METABOLIC DISEASE INHIBITORY EFFECT OF ELAEOCARPUS PETIOLATUS IN CARDIOVASCULAR AND METABOLIC DISEASE MODEL MOUSE

Carotid artery ligation was performed 1 week after injecting AAV-PCSK9 Virus (1×10¹¹ ifu/mL) and Elaeocarpus petiolatus (30 μg/g/day) into the prepared cardiovascular and metabolic disease model mice. After 4 weeks of ligation, it was confirmed whether the model was completed, and gross plaque imaging, plasma lipid profile in serum, and body weight change were measured. As a result of the experiment, as can be seen from the gross plaque imaging results, it was confirmed that the atherosclerotic plaques were reduced in the group treated with Elaeocarpus petiolatus (FIG. 6b). In addition, as can be seen from the plasma lipid profile results, it was confirmed that triglyceride (TG) and low-density lipoprotein cholesterol (LDL-C) were decreased and high-density lipoprotein cholesterol (HDL-C) increased in the group treated with Elaeocarpus petiolatus (FIG. 6c). Further, as a result of comparing the change in body weight after 7 weeks, the body weight was reduced by about 10% in the group treated with Elaeocarpus petiolatus (FIG. 6d).

Based on to the above results, it was confirmed that it was effective in cardiovascular and metabolic diseases such as arteriosclerosis by reducing atherosclerotic plaque, and it was also confirmed that it is effective not only in arteriosclerosis but also in cardiovascular and metabolic diseases such as dyslipidemia as the effects of reducing triglycerides, low-density lipoprotein cholesterol and increasing high-density lipoprotein cholesterol were confirmed.

In addition, the effect of reducing body weight was also confirmed, and thus the prevention effect of obesity due to cardiovascular and metabolic diseases was also confirmed.

EXAMPLE 7. COMPARISON OF CHROMATOGRAMS BETWEEN ELAEOCARPUS PETIOLATUS AND ELAEOCARPUS GANITRUS

UPLC analysis for the qualitative analysis of active indicator substances of Elaeocarpus petiolatus (EP) and Elaeocarpus ganitrus (EG) leaf extracts (extraction solvent: 95% ethanol) was performed by ACQUITY UPLC Ultra Performance equipped with a BEH C18 (2.1 mm×100 mm, 1.7 mm) column tube and a PDA detector (200-600 nm, max plot) with a flow rate of 0.4 mL/min using distilled water containing 0.1% formic acid and acetonitrile (Table 1 and FIG. 7a).

### EXAMPLE 8. COMPARISON OF TNF-A INHIBITORY ACTIVITY OF ELAEOCARPUS PETIOLATUS AND ELAEOCARPUS GANITRUS ON RESISTIN

In order to compare the therapeutic efficacy of cardiovascular and metabolic diseases between plants of the genus Elaeocarpus, the inhibitory effects of TNF-α production by Resistin in THP cells were compared on each extract of Elaeocarpus petiolatus and Elaeocarpus ganitrus, and butanol and water layer as effective fractions (FIG. 7b). It was confirmed that no cytotoxicity was observed at a concentration of 0.6 μg/mL to 20 μg/mL in all three samples for the extracts and fractions of the Elaeocarpus petiolatus and Elaeocarpus ganitrus.

In addition, for the TNF-α production by Resistin in THP-1 cells when the samples were treated at a concentration of 0.6 μg/mL to 20 μg/mL, the TNF-α production was inhibited in a concentration-dependent manner when treated with extracts and effective fractions of Elaeocarpus petiolatus (EP). In contrast, no inhibitory effect was observed for the extract of Elaeocarpus ganitrus (FIG. 7b).

Accordingly, from the results above, it was found that the TNF-α inhibitory activity effect by Resistin in the extract and active fractions of Elaeocarpus petiolatus was more remarkable than that of the extract of Elaeocarpus ganitrus.

In addition, based on the results, not all plants of the genus Elaeocarpus exhibited the same efficacy, but in particular, Elaeocarpus petiolatus has an TNF-α inhibitory activity by Resistin, suggesting that it can be used for the treatment of cardiovascular and metabolic diseases.

### EXAMPLE 9. COMPARISON OF RESISTIN-CAP1 BINDING INHIBITORY ACTIVITY OF COMPOUND DERIVED FROM ELAEOCARPUS PETIOLATUS AND MYRICETIN

Myricetin (Sigma, M6760), which is the parent of EP_5E2 (Myricetin 3-O-α-L-Rha), a compound isolated from Elaeocarpus petiolatus, was purchased, and the binding inhibitory activity of Resistin-CAP1 was measured. As a result, when each sample was treated at a concentration of 25 μM, 50 μM, and 100 μM, the EP_5E2 compound inhibited the binding of Resistin-CAP1 in a concentration-dependent manner, whereas myricetin had no inhibitory activity (FIG. 8).

As a result, it was confirmed that the compound isolated from Elaeocarpus petiolatus of the present invention had an effect different therefrom and from its parent compound, and as a result, since Elaeocarpus petiolatus shows an inhibitory effect of TNF-α activity by Resistin, it was confirmed again that it can be used for the treatment of cardiovascular and metabolic diseases.

From the foregoing, a skilled person in the art to which the present invention pertains will be able to understand that the present invention may be embodied in other specific forms without modifying the technical concepts or essential characteristics of the present invention. In this regard, the exemplary embodiments disclosed herein are only for illustrative purposes and should not be construed as limiting the scope of the present invention. On the contrary, the present invention is intended to cover not only the exemplary embodiments but also various alternatives, modifications, equivalents, and other embodiments that may be included within the spirit and scope of the present invention as defined by the appended claims.

1. A method for treating cardiovascular and metabolic diseases, comprising: administering a composition comprising an Elaeocarpus petiolatus extract, a fraction thereof, or a compound isolated therefrom, as an active ingredient to the subject in need thereof.

2. The method of claim 1, wherein the cardiovascular and metabolic disease is any one selected from the group consisting of hypertension, angina, myocardial infarction, cerebral infarction, stroke, arrhythmia, dyslipidemia, hyperlipidemia, and arteriosclerosis.

3. The method of claim 1, wherein the Elaeocarpus petiolatus extract is extracted from the leaves of Elaeocarpus petiolatus.

4. The method of claim 1, wherein the Elaeocarpus petiolatus extract is prepared by extraction with a solvent selected from the group consisting of water, an alcohol having 1 to 4 carbon atoms, and a mixed solvent thereof.

5. The method of claim 1, wherein the fraction is fractionated with a solvent selected from the group consisting of hexane (n-hexane), ethyl acetate (EA), butanol (n-BuOH), water, and a mixed solvent thereof.

6. The method of claim 1, wherein the compound is selected from the group consisting of methylgallate-O-hexoside, myricetin-3-O-α-L-rhamnoside, ellagic acid, or isorhamnetin-3-O-β-D-hexoside.

7. The method of claim 1, wherein the composition inhibits the binding of Resistin-CAP1.

8. The method of claim 1, wherein the composition inhibits the production of TNF-α.

9. The method of claim 1, wherein the composition inhibits the transcription factor NF-κB.

10. (canceled)

11. A method for improving cardiovascular and metabolic diseases, comprising: administering a food composition comprising an Elaeocarpus petiolatus extract, a fraction thereof, or a compound isolated therefrom, as an active ingredient.

12. (canceled)
